(19) United States
(12) Patent Application Publication
(10) Pub. No.: US 2010/0184816 A1
(43) Pub. Date: Jul. 22, 2010

(54) Method for Reducing the Phytotoxicity of Azoles on Dicotyledonous Plants by Adding Additives

(76) Inventor: Isolde Hauser-Hahn, Leverkusen (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/665,283

(22) PCT Filed: Jun. 10, 2008
(86) PCT No.: PCT/EP08/04610

§ 371 Date: Dec. 17, 2009

(30) Foreign Application Priority Data
Jun. 22, 2007 (EP) 07110836.9

(51) Int. Cl. A01N 43/653; C07C 309/02; A01P 3/00

(52) U.S. Cl. 514/383; 560/149

(57) ABSTRACT

The present invention relates to a method for reducing the phytotoxicity of azoles on dicotyledonous plants by addition of additives. Furthermore, the application relates to novel compositions comprising certain triazole fungicides in combination with additives, in particular alkali metal sulphosuccinates, and their use for controlling unwanted phytopathogenic fungi.

The present invention relates to a method for reducing the phytotoxicity of azoles on dicotyledonous plants by addition of additives. Furthermore, the application relates to novel compositions comprising certain triazole fungicides in combination with additives, in particular alkali metal sulphosuccinates, and their use for controlling unwanted phytopathogenic fungi.

Triazole fungicides which inhibit ergosterol biosynthesis are economically important active compounds and are widely used in crop plants such as wheat, barley, leguminous plants, vegetables and in the cultivation of fruits. However, these fungicides may cause phytotoxic damage to some plants when used at their customary application rates for controlling unwanted fungi. This phytotoxic effect can be observed in particular in stress situations such as drought or when the triazole fungicides are used in combination with penetration promoters.

To minimize the phytotoxic effect, WO 2007/028388 proposes a certain formulation which, in addition to the triazole fungicides as active compound, comprises various further components, such as solvents and surfactants.

It is already known from DE-A 24 08 662 that certain alkali metal sulphosuccinates (for example sodium dioctylsulphosuccinate in alcohol/water) can be used to reduce unwanted damage to the plants when applying fungicides, for example ethirimol (a pyrimidine derivative).

It is furthermore known from FR-A 2 636 503 that sodium dioctylsulphosuccinate can be used to increase the efficiency of the triazole fungicide bitertanol.

Finally, EP-A 0 897 665 discloses the combination of tebuconazole with alkali metal sulphosuccinates which, compared to earlier tebuconazole formulations, has a higher efficacy.

Since the environmental and economic requirements imposed on modern-day crop protection compositions are continually increasing, with regard, for example, to the spectrum of action, toxicity, selectivity, application rate, formation of residues, and favourable preparability, and since, furthermore, there may be problems, for example, with resistances, a constant task is to develop new compositions which in some areas at least help to overcome the abovementioned disadvantages.

The present invention provides a method which in some aspects at least achieves the stated objective. Moreover, active compound combinations or compositions are provided which likewise in some aspects at least achieve the stated objective.

Surprisingly, it has now been found that addition of sulphosuccinates to triazole fungicides reduces or even completely suppresses their phytotoxic action.

Accordingly, the present invention relates to a method for reducing the phytotoxicity of triazole fungicides on dicotyledonous plants, characterized in that sulphosuccinates of the general formula (I)

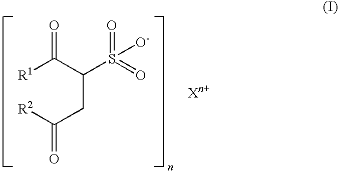

in which
R¹ and R² independently of one another represent hydroxyl, alkoxy or cycloalkoxy or represent the group

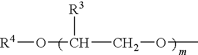

where at least one radical R¹ or R² does not represent hydroxyl,
R³ represents hydrogen or methyl,
R⁴ represents hydrogen, alkyl or aryl,
m represents an integer from 0 to 100,
X represents an alkali metal, where n represents 1, or an alkaline earth metal, where n represents 2
   are added to the fungicidal compositions, which comprise at least one triazole fungicide.

In the method according to the invention, use is preferably made of sulphosuccinates of the formula (I) where the radicals have the following meaning:
R¹ and R² independently of one another preferably represent hydroxyl, C₁-C₁₆-alkoxy or C₅-C₈-cycloalkoxy or represent the group

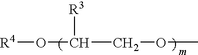

where at least one radical R¹ or R² does not represent hydroxyl.
R¹ and R² independently of one another particularly preferably represent hydroxyl, C₃-C₁₃-alkoxy or C₅-C₆-cycloalkoxy or represent the group

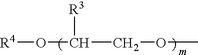

where at least one radical R¹ or R² does not represent hydroxyl.
R¹ and R² independently of one another very particularly preferably represent hydroxyl, propoxy, butoxy, pentyloxy, hexyloxy, octyloxy, decyloxy, dodecyloxy, tridecyloxy, cyclopentyloxy or cyclohexyloxy or represent the group

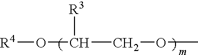

where at least one radical R¹ or R² does not represent hydroxyl.
R¹ and R² independently of one another especially preferably represent hydroxyl, isopropyloxy, isobutyloxy, pentyloxy, hexyloxy, octyloxy, 2-ethylhexyloxy, dodecyloxy, tridecyloxy or cyclohexyloxy or represent the group

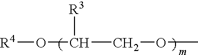

where at least one radical R¹ or R² does not represent hydroxyl.

With emphasis, R¹ and R² do not both represent hydroxyl (diester).
R³ preferably represents hydrogen.
R³ also preferably represents methyl.
R⁴ preferably represents hydrogen, C₁-C₆-alkyl, phenyl or benzyl.
R⁴ particularly preferably represents hydrogen, C₁-C₄-alkyl, phenyl or benzyl.
R⁴ very particularly preferably represents hydrogen, methyl, ethyl, n-propyl, isopropyl, n-, i-, s-, t-butyl.
R⁴ especially preferably represents hydrogen or methyl.
m preferably represents a number from 0 to 50.
m particularly preferably represents a number from 0 to 30.
m very particularly preferably represents a number from 1 to 25.
X preferably represents sodium, potassium or ammonium, where n represents 1, or represents magnesium or calcium, where n represents 2.
X particularly preferably represents sodium, potassium or ammonium, where n represents 1.
X very particularly preferably represents sodium.

Preference is given to using sulphosuccinates of the formula (I) in which the radical definitions have the meanings mentioned as being preferred.

Particular preference is given to using sulphosuccinates of the formula (I) in which the radical definitions have the meanings mentioned as being particularly preferred.

Very particular preference is given to using sulphosuccinates of the formula (I) in which the radical definitions have the meanings mentioned as being very particularly preferred.

Special preference is given to using sulphosuccinates of the formula (I) in which the radical definitions have the meanings mentioned as being especially preferred.

Specific mention may be made of the following sulphosuccinates of the formula (I):

sodium diisopropylsulphosuccinate,
sodium diisobutylsulphosuccinate,
sodium dipentylsulphosuccinate,
sodium dihexylsulphosuccinate,
sodium dioctylsulphosuccinate,
sodium di(2-ethylhexyl)sulphosuccinate,
sodium didodecylsulphosuccinate,
sodium tridecylsulphosuccinate,
sodium dicyclohexylsulphosuccinate.

From among these, the sodium di(2-ethylhexyl)sulphosuccinate may be used with even more preference.

The sulphosuccinates of the formula (I) are known. They can be used both in a liquid and in a solid application form. It is also possible to use mixtures of different sulphosuccinates of the formula (I).

The sulphosuccinates of the formula (I) can be used within a relatively wide concentration range. In general, the amounts used for the ready-to-use spray liquor are from 0.05 to 5 g/l, preferably from 0.05 to 1 g/l, particularly preferably from 0.2 to 0.8 g/l. Application in the range of from 0.1 to 2 g/l is also possible.

The addition of the sulphosuccinates of the formula (I) may take place at various points in time. Firstly, it is possible to add the sulphosuccinates already to the active compound formulation. Secondly, it is possible to mix the sulphosuccinates shortly before the application to the plant with customary formulations of the triazole fungicides (tank mix).

The following compounds may be mentioned as being preferred as triazole fungicides for use in the method according to the invention: azaconazole, bitertanol, bromuconazole, cyproconazole, difenoconazole, diniconazole, diniconazole-M, epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, simeconazole, tebuconazole, tetraconazole, triadimefon, triadimenol, triticonazole, and in each case salts thereof. From among these, particularly preference is given to cyproconazole, epoxiconazole, metconazole, prothioconazole and tebuconazole.

Very particular preference is given to using tebuconazole. Very particular preference is also given to using prothioconazole. Very particular preference is also given to using epoxiconazole. Very particular preference is also given to using cyproconazole.

The triazole fungicides mentioned are known (cf. The Pesticide Manual, 13th Edition, British Crop Protection Council, 2003).

In the method according to the invention, use is made of at least one of the triazole fungicides mentioned. However, it is also possible to use mixtures of the triazole fungicides mentioned, for example binary mixtures and ternary mixtures. The mixtures of tebuconazole and prothioconazole, tebuconazole and epoxiconazole, prothioconazole and epoxiconazole may be mentioned by way of example, with the mixture of tebuconazole and prothioconazole being preferred.

Furthermore, in addition to a triazole fungicide it is also possible to use another agrochemical active compound. Suitable mixing partners are, for example, the following compounds:

### Fungicides:

1) Nucleic acid synthesis inhibitors: for example benalaxyl, benalaxyl-M, bupirimate, clozylacon, dimethirimol, ethirimol, furalaxyl, hymexazol, mefenoxam, metalaxyl, metalaxyl-M, ofurace, oxadixyl, oxolinic acid;
2) mitosis and cell division inhibitors: for example benomyl, carbendazim, diethofencarb, ethaboxam, fuberidazole, pencycuron, thiabendazole, thiophanate-methyl, zoxamide;
3) respiration inhibitors (inhibitors of the respiratory chain):
3.1) inhibitors which act on complex I of the respiratory chain: for example diflumetorim;
3.2) inhibitors which act on complex II of the respiratory chain: for example boscalid, carboxin, fenfuram, flutolanil, furametpyr, furmecyclox, mepronil, oxycarboxin, penthiopyrad, thifluzamide;
3.3) inhibitors which act on complex III of the respiratory chain: for example amisulbrom, azoxystrobin, cyazofamid, dimoxystrobin, enestrobin, famoxadone, fenamidone, fluoxastrobin, kresoxim-methyl, metominostrobin, orysastrobin, picoxystrobin, pyraclostrobin, trifloxystrobin;
4) decouplers: for example dinocap, fluazinam, meptyldinocap;
5) ATP production inhibitors: for example fentin acetate, fentin chloride, fentin hydroxide, silthiofam;
6) amino acid and protein biosynthesis inhibitors: for example andoprim, blasticidin-S, cyprodinil, kasugamycin, kasugamycin hydrochloride hydrate, mepanipyrim, pyrimethanil;
7) signal transduction inhibitors: for example fenpiclonil, fludioxonil, quinoxyfen;
8) lipid and membrane synthesis inhibitors: for example biphenyl, chlozolinate, edifenphos, etridiazole, iodocarb, iprobenfos, iprodione, isoprothiolane, procymidone, propamocarb, propamocarb hydrochloride, pyrazophos, tolclofos-methyl, vinclozolin;
9) ergosterol biosynthesis inhibitors: for example aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulphate, imibenconazole, ipconazole, metconazole, myclobutanil, naftifine, nuarimol, oxpoconazole, paclobutrazol, pefurazoate, penconazole, prochloraz, propiconazole, prothioconazole, pyributicarb, pyrifenox, simeconazole, spiroxamine, tebuconazole, terbinafine, tetraconazole, triadimefon, triadimenol, tridemorph, triflumizole, triforine, triticonazole, uniconazole, viniconazole, voriconazole;
10) cell wall synthesis inhibitors: for example benthiavalicarb, dimethomorph, flumorph, iprovalicarb, mandipropamid, polyoxins, polyoxorim, validamycin A;
11) melanin biosynthesis inhibitors: for example carpropamid, diclocymet, fenoxanil, phthalide, pyroquilon, tricyclazole;
12) resistance inductors: for example acibenzolar-5-methyl, probenazole, tiadinil;
13) compounds with multi-site activity: for example Bordeaux mixture, captafol, captan, chlorothalonil, copper naphthenate, copper oxide, copper oxychloride, copper preparations such as, for example, copper hydroxide, copper sulphate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propineb, sulphur and sulphur preparations such as, for example, calcium polysulphide, thiram, tolylfluanid, zineb, ziram;
14) a compound selected from the following enumeration: (2E)-2-(2-{[6-(3-chloro-2-methylphenoxy)-5-fluoropyrimidin-4-yl]oxy}phenyl)-2-(methoxyimino)-N-methylacetamide, (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, 1-(4-chlorophenyl)-2-(1H-1,2,4-triazol-1-yl)cycloheptanol, 1-[(4-methoxyphenoxy)methyl]-2,2-dimethylpropyl-1H-imidazole-1-carboxylate, 1-methyl-N-[2-(1,1,2,2-tetrafluoroethoxy)phenyl]-3-(trifluoromethyl)-1H-pyrazole-4-carboxamide, 2,3,5,6-tetrachloro-4-(methylsulphonyl)pyridine, 2-butoxy-6-iodo-3-propyl-4H-chromen-4-one, 2-chloro-N-(1,1,3-trimethyl-2,3-dihydro-1H-inden-4-yl)nicotinamide, 2-phenylphenol and salts thereof, 3-(difluoromethyl)-1-methyl-N-[2-(1,1,2,2-tetrafluoroethoxy)phenyl]-1H-pyrazole-4-carboxamide, 3-(difluoromethyl)-N-[(9R)-9-isopropyl-1,2,3,4-tetrahydro-1,4-methanonaphthalen-5-yl]-1-methyl-1H-pyrazole-4-carboxamide, 3-(difluoromethyl)-N-[(9S)-9-isopropyl-1,2,3,4-tetrahydro-1,4-methanonaphthalen-5-yl]-1-methyl-1H-pyrazole-4-carboxamide, 3-(difluoromethyl)-N-[4′-(3,3-dimethylbut-1-yn-1-yl)biphenyl-2-yl]-1-methyl-1H-pyrazole-4-carboxamide, 3,4,5-trichloropyridine-2,6-dicarbonitrile, 3-[5-(4-chlorophenyl)-2,3-dimethylisoxazolidin-3-yl]pyridine, 3-chloro-5-(4-chlorophenyl)-4-(2,6-difluorophenyl)-6-methylpyridazine, 4-(4-chlorophenyl)-5-(2,6-difluorophenyl)-3,6-dimethylpyridazine, 5-chloro-7-(4-methylpiperidin-1-yl)-6-(2,4,6-trifluorophenyl)[1,2,4]triazolo[1,5-a]pyrimidine, 8-hydroxyquinoline sulphate, benthiazole, bethoxazin, capsimycin, carvone, chinomethionat, cufraneb, cyflufenamid, cymoxanil, dazomet, debacarb, dichlorophen, diclomezine, dicloran, difenzoquat, difenzoquat methylsulphate, diphenylamine, ecomate, ferimzone, flumetover, fluopicolide, fluoroimide, flusulphamide, fosetyl-aluminium, fosetyl-calcium, fosetyl-sodium, hexachlorobenzene, irumamycin, isotianil, methasulphocarb, methyl (2E)-2-{2-[({cyclopropyl[(4-methoxyphenyl)-imino]methyl}thio)methyl]phenyl}-3-methoxyacrylate, methyl 1-(2,2-dimethyl-2,3-dihydro-1H-inden-1-yl)-1H-imidazole-5-carboxylate, methyl isothiocyanate, metrafenone, mildiomycin, N-[2-(1,3-dimethylbutyl)phenyl]-5-fluoro-1,3-dimethyl-1H-pyrazole-4-carboxamide, N-(3′,4′-dichloro-5-fluorobiphenyl-2-yl)-3-(difluoromethyl)-1-methyl-1H-pyrazole-4-carboxamide (bixafen), N-(3-ethyl-3,5,5-trimethylcyclohexyl)-3-(formylamino)-2-hydroxybenzamide, N-(4-chloro-2-nitrophenyl)-N-ethyl-4-methylbenzenesulphonamide, N-(4-chlorobenzyl)-3-[3-methoxy-4-(prop-2-yn-1-yloxy)phenyl]propanamide, N-[(4-chlorophenyl)(cyano)methyl]-3-[3-methoxy-4-(prop-2-yn-1-yloxy)phenyl]propanamide, N-[(5-bromo-3-chloropyridin-2-yl)methyl]-2,4-dichloronicotinamide, N-[1-(5-bromo-3-chloropyridin-2-yl)ethyl]-2,4-dichloronicotinamide, N-[1-(5-bromo-3-chloropyridin-2-yl)ethyl]-2-fluoro-4-iodonicotinamide, N-[2-(4-{[3-(4-chlorophenyl)prop-2-yn-1-yl]oxy}-3-methoxyphenyl)ethyl]-N-(methylsulphonyl)valinamide, N-{(Z)-[(cyclopropylmethoxy)imino][6-(difluoromethoxy)-2,3-difluorophenyl]methyl}-2-phenylacetamide, N-{2-[1,1′-bi(cyclopropyl)-2-yl]phenyl}-3-(difluoromethyl)-1-methyl-1H-pyrazole-4-carboxamide, N-{2-[3-chloro-5-(trifluoromethyl)pyridin-2-yl]ethyl}-2-(trifluoromethyl)benzamide (fluopyram), natamycin, N-ethyl-N-methyl-N′-{2-methyl-5-(trifluoromethyl)-4-[3-(trimethylsilyl)propoxy]phenyl}imidoformamide, N-ethyl-N-methyl-N′-{2-methyl-5-(difluoromethyl)-4-[3-(trimethylsilyl)propoxy]phenyl}imidoformamide, nickel dimethyldithiocarbamate, nitrothalisopropyl, O-{1-[(4-methoxyphenoxy)methyl]-2,2-dimethylpropyl}1H-imidazole-1-carbothioate, octhilinone, oxamocarb, oxyfenthiin, pentachlorophenol and salts, phosphoric acid and salts thereof, piperalin, propamocarb-fosetylate, propanosine-sodium, proquinazid, pyribencarb, pyrroinitrins, quintozene, S-allyl 5-amino-2-isopropyl-4-(2-methylphenyl)-3-oxo-2,3-dihydro-1H-pyrazole-1-carbothioate, tecloftalam, tecnazene, triazoxide, trichlamide, valiphenal, zarilamid.

### Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

### Insecticides/Acaricides/Nematicides:

### 1. Acetylcholinesterase (AChE) Inhibitors

1.1 carbamates (for example alanycarb, aldicarb, aldoxycarb, allyxycarb, aminocarb, azamethiphos, bendiocarb, benfuracarb, bufencarb, butacarb, butocarboxim, butoxycarboxim, carbaryl, carbofuran, carbosulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, triazamate, trimethacarb, XMC, xylylcarb)
1.2 organophosphates (for example acephate, azamethiphos, azinphos (-methyl, -ethyl), bromophosethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulphone, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion)

### 2. Sodium Channel Modulators/Blockers of Voltage-Gated Sodium Channels

2.1 pyrethroids (for example acrinathrin, allethrin (d-cis-trans, d-trans), beta-cyfluthrin, bifenthrin, bioallethrin, bioallethrin-S-cyclopentyl-isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, DDT, deltamethrin, empenthrin (1R-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, tefluthrin, terallethrin, tetramethrin (1R-isomer), tralomethrin, transfluthrin, ZXI 8901, pyrethrins (pyrethrum))
2.2 oxadiazines (for example indoxacarb)

### 3. Acetylcholine Receptor Agonists/Antagonists

3.1 chloronicotinyls/neonicotinoids (for example acetamiprid, clothianidin, dinotefuran, imidacloprid, nitenpyram, nithiazine, thiacloprid, thiamethoxam)
3.2 nicotine, bensultap, cartap

### 4. Acetylcholine Receptor Modulators

4.1 spinosyns (for example spinosad)

### 5. Antagonists of GABA-Gated Chloride Channels

5.1 cyclodiene organochlorines (for example camphechlor, chlordane, endosulfan, gamma-HCH, HCH, heptachlor, lindane, methoxychlor)
5.2 fiproles (for example acetoprole, ethiprole, fipronil, vaniliprole)

### 6. Chloride Channel Activators

6.1 mectins (for example abamectin, avermectin, emamectin, emamectin-benzoate, ivermectin, milbemectin, milbemycin)

### 7. Juvenile Hormone Mimetics

(for example diofenolan, epofenonane, fenoxycarb, hydroprene, kinoprene, methoprene, pyriproxifen, triprene)

### 8. Ecdyson Agonists/Disruptors

8.1 diacylhydrazines (for example chromafenozide, halofenozide, methoxyfenozide, tebufenozide)

### 9. Chitin Biosynthesis Inhibitors

9.1 benzoylureas (for example bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron)
9.2 buprofezin
9.3 cyromazine

### 10. Inhibitors of Oxidative Phosphorylation, ATP Disruptors

10.1 diafenthiuron
10.2 organotins (for example azocyclotin, cyhexatin, fenbutatin-oxide)

### 11. Decouplers of Oxidative Phosphorylation Acting by Interrupting the H-Proton Gradient

11.1 pyrroles (for example chlorfenapyr)
11.2 dinitrophenols (for example binapacryl, dinobuton, dinocap, DNOC)

### 12. Site-I Electron Transport Inhibitors

12.1 METIs (for example fenazaquin, fenpyroximate, pyrimidifen, pyridaben, tebufenpyrad, tolfenpyrad)
12.2 hydramethylnone
12.3 dicofol

### 13. Site-II Electron Transport Inhibitors

13.1 rotenone

### 14. Site-III Electron Transport Inhibitors

14.1 acequinocyl, fluacrypyrim

### 15. Microbial Disruptors of the Insect Gut Membrane

Bacillus thuringiensis strains

### 16. Inhibitors of Fat Synthesis

16.1 tetronic acids (for example spirodiclofen, spiromesifen)
16.2 tetramic acids [for example 3-(2,5-dimethylphenyl)-8-methoxy-2-oxo-1-azaspiro[4.5]dec-3-en-4-yl ethyl carbonate (alias: carbonic acid, 3-(2,5-dimethylphenyl)-8-methoxy-2-oxo-1-azaspiro[4.5]dec-3-en-4-yl ethyl ester, CAS Reg. No.: 382608-10-8) and carbonic acid, cis-3-(2,5-dimethylphenyl)-8-methoxy-2-oxo-1-azaspiro[4.5]dec-3-en-4-yl ethyl ester (CAS Reg. No.: 203313-25-1)]

### 17. Carboxamides

(for example flonicamid)

### 18. Octopaminergic Agonists

(for example amitraz)

### 19. Inhibitors of Magnesium-Stimulated ATPase

(for example propargite)

### 20. Ryanodin Receptor Agonists

20.1 benzoic acid dicarboxamides [for example N²-[1,1-dimethyl-2-(methylsulphonyl)ethyl]-3-iodo-N¹-[2-methyl-4-[1,2,2,2-tetrafluoro-1-(trifluoromethyl)ethyl]phenyl]-1,2-benzenedicarboxamide (CAS Reg. No.: 272451-65-7), flubendiamide]
20.2 anthranilamides (for example DPX E2Y45=3-bromo-N-{4-chloro-2-methyl-6-[(methylamino)carbonyl]phenyl}-1-(3-chloropyridin-2-yl)-1H-pyrazole-5-carboxamide)

### 21. Nereistoxin Analogues

(for example thiocyclam hydrogen oxalate, thiosultap-sodium)

### 22. Biologicals, Hormones or Pheromones

(for example azadirachtin, Bacillus spec., Beauveria spec., Codlemone, Metarrhizium spec., Paecilomyces spec., Thuringiensin, Verticillium spec.)
23. Active Compounds with Unknown or Unspecific Mechanisms of Action
23.1 fumigants (for example aluminium phosphide, methyl bromide, sulphuryl fluoride)
23.2 selective antifeedants (for example cryolite, flonicamid, pymetrozine)
23.3 mite growth inhibitors (for example clofentezine, etoxazole, hexythiazox)
23.4 amidoflumet, benclothiaz, benzoximate, bifenazate, bromopropylate, buprofezin, chinomethionat, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin, furthermore the compound 3-methylphenyl propylcarbamate (Tsumacide Z), the compound 3-(5-chloro-3-pyridinyl)-8-(2,2,2-trifluoroethyl)-8-azabicyclo[3.2.1]octane-3-carbonitrile (CAS Reg. No. 185982-80-3) and the corresponding 3-endo-isomer (CAS Reg. No. 185984-60-5) (cf. WO 96/37494, WO 98/25923), and preparations which comprise insecticidally active plant extracts, nematodes, fungi or viruses.

A mixture with other known active compounds, such as herbicides or with fertilizers including foliar fertilizers and growth regulators, safeners and/or semiochemicals, is also possible.

Preference is given to using combinations with the fungicides mentioned, particularly preferably those with inhibitors at complex III of the respiratory chain selected from the group consisting of azoxystrobin, dimoxystrobin, enestrobin, fluoxastrobin, kresoxim-methyl, metominostrobin, orysastrobin, picoxystrobin, pyraclostrobin and trifloxystrobin. Preference is given to combinations with azoxystrobin, pyraclostrobin, fluoxastrobin and trifloxystrobin, particularly preferably with trifloxystrobin. Specifically, preference is given to the combinations of tebuconazole and trifloxystrobin, tebuconazole and fluoxastrobin, tebuconazole and azoxystrobin, tebuconazole and pyraclostrobin, prothioconazole and trifloxystrobin, prothioconazole and azoxystrobin and also prothioconazole and pyraclostrobin.

The dicotyledonous plants to be treated according to the invention are to be understood as meaning all plants from the class Dicotyledoneae. These include, for example, the following plants: useful plants, such as cotton, flax, grapevines, fruit, such as pomaceous fruit, but also stone fruit, vegetables, such as cucumbers, tomatoes, cabbage, and useful plants, such as soya beans, peanuts, potatoes, and also ornamental plants, such as annual and/or perennial trees and shrubs. The plants to be treated preferably include representatives of Rosaceae sp. (for example pomaceous fruit, such as apple, pear and quince, but also stone fruit, such as apricots, cherries, almonds, plums and peaches, and soft fruit, such as strawberries, blackberries, blackcurrants, raspberries), Juglandaceae (walnut), Betulaceae (hazelnut), Anacardiaceae (cashew nut; pistachio), Fagaceae (edible chestnut), Moraceae sp. (mulberry tree), Oleaceae sp., Actimidaceae (kiwi), Lauraceae sp., Rubiaceae sp. (for example coffee), Theaceae sp., Sterculiceae (cocoa), Rutaceae sp. (for example lemons, oranges and grapefruit), Umbelliferae sp., Cucurbitaceae sp. (for example cucumber, melon), Fabaceae sp. (for example peas, soya beans, common beans, broad beans, lentils, peanuts), Asteraceae sp. (for example lettuce, sunflower, Jerusalem artichoke), Brassicaceae sp. (for example white cabbage, red cabbage, broccoli, cauliflower, brussel sprouts, pak choi, kohlrabi, radish and also oil seed rape, mustard, horseradish and cress), Solanaceae sp. (for example tomatoes, potatoes, tobacco, bell peppers), Chenopodiaceae sp. (for example sugar beet, fodder beet, Swiss chard, beetroot); however, useful plants and ornamental plants in garden and forests; and also in each case genetically modified varieties of these plants. Particular preference is given to treating soya bean, cotton, peanuts, pomaceous fruit and stone fruit, and also grapevines.

A further aspect of the invention are novel combinations comprising
(a) exactly one triazole fungicide selected from the group consisting of azaconazole, bromuconazole, cyproconazole, difenoconazole, diniconazole, diniconazole-M, epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, simeconazole, tetraconazole, triadimefon, triadimenol, triticonazole, and in each case salts thereof,
(b) a sulphosuccinate of the formula (I)

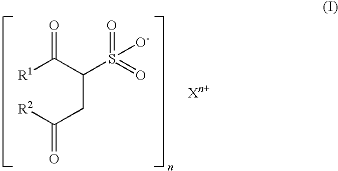

in which
R¹ and R² independently of one another represent hydroxyl, alkoxy or cycloalkoxy or represent the group

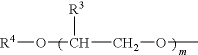

where at least one radical R¹ or R² does not represent hydroxyl,
R³ represents hydrogen or methyl,
R⁴ represents hydrogen, alkyl or aryl,
m represents an integer from 0 to 100,
X represents an alkali metal, where n represents 1, or an alkaline earth metal, where n represents 2.

From among these, preference is given to compositions in which the triazole fungicide is selected from the group consisting of cyproconazole, epoxiconazole, metconazole and prothioconazole. Very particular preference is given to compositions comprising prothioconazole. Very particular preference is also given to compositions comprising epoxiconazole. Very particular preference is also given to compositions comprising cyproconazole.

Preferred, particularly preferred and very particularly preferred are furthermore compositions in which R¹, R², R³, R⁴, n, m and X have the preferred, particularly preferred and very particularly preferred meanings, respectively, given above. Especially preferred are compositions comprising at least one of the sulphosuccinates mentioned above by name, especially sodium di(2-ethylhexyl)sulphosuccinate. It is also possible to use mixtures of different sulphosuccinates of the formula (I).

The compositions according to the invention comprise at least one of the triazole fungicides mentioned under (a). However, it is also possible to use mixtures of the triazole fungicides mentioned under (a), suitable additional mixing partners being bitertanol and tebuconazole, for example binary mixtures and ternary mixtures. The mixtures of tebuconazole and prothioconazole, tebuconazole and epoxiconazole, prothioconazole and epoxiconazole may be mentioned by way of example, with the mixture of tebuconazole and prothioconazole being preferred.

In addition to the triazole fungicides mentioned, the compositions according to the invention may comprise a further agrochemically active compound, such as the fungicides, bactericides, insecticides, acaricides and nematicides mentioned above, and also other known active compounds, for example herbicides or fertilizers including foliar fertilizers, and growth regulators, safeners and/or semiochemicals.

Preferably, the compositions according to the invention comprise combinations with the fungicides mentioned, particularly preferably those with inhibitors at complex III of the respiratory chain selected from the group consisting of azoxystrobin, dimoxystrobin, enestrobin, fluoxastrobin, kresoxim-methyl, metominostrobin, orysastrobin, picoxystrobin, pyraclostrobin and trifloxystrobin. Preferably, the compositions according to the invention comprise combinations with azoxystrobin, pyraclostrobin, fluoxastrobin and trifloxystrobin, particularly preferably with trifloxystrobin. Specifically, the compositions according to the invention comprise the combinations of tebuconazole and trifloxystrobin, tebuconazole and fluoxastrobin, tebuconazole and azoxystrobin, tebuconazole and pyraclostrobin, prothioconazole and trifloxystrobin, prothioconazole and azoxystrobin and also prothioconazole and pyraclostrobin.

In the compositions according to the invention, in general from 0.05 to 10, preferably from 0.1 to 2, particularly preferably from 0.2 to 1, parts by weight of sulphosuccinate of the formula (I) are present per part by weight of triazole fungicide.

According to the invention, the term “combination” means various possible combinations of triazole fungicide and sulphosuccinate, such as, for example, ready mixes, tank mixes (which is to be understood as meaning application and spray mixtures prepared prior to application from the formulation of triazole fungicide and sulphosuccinate by mixing and diluting) or combinations thereof.

The compositions according to the invention are preferably fungicidal compositions comprising agriculturally suitable carriers or extenders.

According to the invention, carrier is to be understood as meaning a natural or synthetic, organic or inorganic substance which is mixed or combined with the active compounds for better applicability, in particular for application to plants or plant parts or seeds. The carrier, which may be solid or liquid, is generally inert and should be suitable for use in agriculture.

Suitable solid carriers are: for example ammonium salts and natural ground minerals, such as kaolins, clays, talc, chalk, quartz, attapulgite, montmorillonite or diatomaceous earth, and ground synthetic minerals, such as finely divided silica, alumina and natural or synthetic silicates, resins, waxes, solid fertilizers, water, alcohols, especially butanol, organic solvents, mineral oils and vegetable oils, and also derivatives thereof. It is also possible to use mixtures of such carriers. Solid carriers suitable for granules are: for example crushed and fractionated natural minerals, such as calcite, marble, pumice, sepiolite, dolomite, and also synthetic granules of inorganic and organic meals and also granules of organic material, such as sawdust, coconut shells, maize cobs and tobacco stalks. Suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, and also protein hydrolysates. Suitable dispersants are: for example lignosulphite waste liquors and methylcellulose.

Suitable liquefied gaseous extenders or carriers are liquids which are gaseous at ambient temperature and under atmospheric pressure, for example aerosol propellants, such as butane, propane, nitrogen and carbon dioxide.

Tackifiers, such as carboxymethylcellulose and natural and synthetic polymers in the form of powders, granules and latices, such as gum arabic, polyvinyl alcohol, polyvinyl acetate, or else natural phospholipids, such as cephalins and lecithins and synthetic phospholipids can be used in the formulations. Other possible additives are mineral and vegetable oils.

If the extender used is water, it is also possible for example, to use organic solvents as auxiliary solvents. Suitable liquid solvents are essentially: aromatic compounds, such as xylene, toluene or alkylnaphthalenes, chlorinated aromatic compounds or chlorinated aliphatic hydrocarbons, such as chlorobenzenes, chloroethylenes or methylene chloride, aliphatic hydrocarbons, such as cyclohexane or paraffins, for example mineral oil fractions, mineral and vegetable oils, alcohols, such as butanol or glycol, and also ethers and esters thereof, ketones, such as acetone, methyl ethyl ketone, methyl isobutyl ketone or cyclohexanone, strongly polar solvents, such as dimethylformamide and dimethyl sulphoxide, and also water.

The compositions according to the invention may additionally comprise further components, such as, for example, surfactants. Suitable surfactants are emulsifiers, dispersants or wetting agents having ionic or nonionic properties, or mixtures of these surfactants. Examples of these are salts of polyacrylic acid, salts of lignosulphonic acid, salts of phenolsulphonic acid or naphthalenesulphonic acid, polycondensates of ethylene oxide with fatty alcohols or with fatty acids or with fatty amines, substituted phenols (preferably alkylphenols or arylphenols), salts of sulphosuccinic esters, taurine derivatives (preferably alkyl taurates), phosphoric esters of polyethoxylated alcohols or phenols, fatty esters of polyols, and derivatives of the compounds containing sulphates, sulphonates and phosphates. The presence of a surfactant is required if one of the active compounds and/or one of the inert carriers is insoluble in water and when the application takes place in water. The proportion of surfactants is between 5 and 40 percent by weight of the composition according to the invention.

It is possible to use colorants such as inorganic pigments, for example iron oxide, titanium oxide, Prussian blue, and organic dyes, such as alizarin dyes, azo dyes and metal phthalocyanine dyes, and trace nutrients, such as salts of iron, manganese, boron, copper, cobalt, molybdenum and zinc.

If appropriate, other additional components may also be present, for example protective colloids, binders, adhesives, thickeners, thixotropic substances, penetrants, stabilizers, sequestring agents, complex formers. In general, the active compounds can be combined with any solid or liquid additive customarily used for formulation purposes.

In general, the compositions according to the invention comprise between 0.05 and 99 percent by weight of the active compound combination according to the invention, preferably between 10 and 70 percent by weight, particularly preferably between 20 and 50 percent by weight, very particularly preferably 25 percent by weight.

The active compound combinations or compositions according to the invention can be used as such or, depending on their respective physical and/or chemical properties, in the form of their formulations or the use forms prepared therefrom, such as aerosols, capsule suspensions, cold-fogging concentrates, warm-fogging concentrates, encapsulated granules, fine granules, flowable concentrates for the treatment of seed, ready-to-use solutions, dustable powders, emulsifiable concentrates, oil-in-water emulsions, water-in-oil emulsions, macrogranules, microgranules, oil-dispersible powders, oil-miscible flowable concentrates, oil-miscible liquids, foams, pastes, pesticide-coated seed, suspension concentrates, suspoemulsion concentrates, soluble concentrates, suspensions, wettable powders, soluble powders, dusts and granules, water-soluble granules or tablets, water-soluble powders for the treatment of seed, wettable powders, natural products and synthetic substances impregnated with active compound, and also microencapsulations in polymeric substances and in coating materials for seed, and also ULV cold-fogging and warm-fogging formulations.

The formulations mentioned can be prepared in a manner known per se, for example by mixing the active compounds or the active compound combinations with at least one customary extender, solvent and/or diluent, emulsifier, dispersant and/or binder or fixing agent, wetting agent, water-repellent, if appropriate siccatives and UV stabilizers and, if appropriate, dyes and pigments, antifoams, preservatives, secondary thickeners, adhesives, gibberellins, and also further processing auxiliaries.

The treatment according to the invention of the plants and plant parts with the active compound combinations or compositions is carried out directly or by action on their surroundings, habitat or storage space using customary treatment methods, for example by dipping, spraying, atomizing, irrigating, evaporating, dusting, fogging, broadcasting, foaming, painting, spreading-on, watering (drenching), drip irrigating and, in the case of propagation material, in particular in the case of seeds, furthermore as a powder for dry seed treatment, a solution for seed treatment, a water-soluble powder for slurry treatment, by incrusting, by coating with one or more coats, etc.

The compositions according to the invention do not only comprise ready-to-use compositions which can be applied with suitable apparatus to the plant or the seed, but also commercial concentrates which have to be diluted with water prior to use.

The active compound combinations according to the invention can be present in commercial formulations and in the use forms prepared from these formulations as a mixture with other active compounds, such as insecticides, attractants, sterilants, bactericides, acaricides, nematicides, fungicides, growth regulators or herbicides.

The active compound combinations or compositions according to the invention have strong microbicidal activity and can be used for controlling unwanted microorganisms, such as fungi and bacteria, in crop protection and in the protection of materials.

In crop protection, fungicides can be used for controlling Plasmodiophoromycetes, Oomycetes, Chytridiomycetes, Zygomycetes, Ascomycetes, Basidiomycetes and Deuteromycetes.

In crop protection, bactericides can be used for controlling Pseudomonadaceae, Rhizobiaceae, Enterobacteriaceae, Corynebacteriaceae and Streptomycetaceae.

The fungicidal compositions according to the invention can be used for the curative or protective control of phytopathogenic fungi. Accordingly, the invention also relates to curative and protective methods for controlling phytopathogenic fungi using the active compound combinations or compositions according to the invention, which are applied to the seed, the plant or plant parts, the fruit or the soil in which the plants grow.

According to the invention, it is possible to treat all plants and parts of plants. Plants are to be understood here as meaning all plants and plant populations, such as wanted and unwanted wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by varietal property rights. Parts of plants are to be understood as meaning all above-ground and below-ground parts and organs of the plants, such as shoot, leaf, flower and root, examples which may be mentioned being leaves, needles, stems, trunks, flowers, fruit bodies, fruits and seeds and also roots, tubers and rhizomes. Plant parts also include harvested material and vegetative and generative propagation material, for example seedlings, tubers, rhizomes, cuttings and seeds.

For plants which may be treated according to the invention, reference may be made to the dicotyledonous plants mentioned above.

The method according to the invention for controlling phytopathogenic fungi can also be employed for treating genetically modified organisms, for example plants or seeds. Genetically modified plants are plants whose genome has, stably integrated, a certain heterologous gene coding for a certain protein. Here, “heterologous gene” is meant to be understood as a gene which confers novel agronomical properties on the transformed plant, or a gene which improves the agronomical quality of the modified plant.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The terms “parts”, “parts of plants” and “plant parts” have been explained above. Particularly preferably, plants of the plant cultivars which are in each case commercially available or in use are treated according to the invention.

Depending on the plant species or plant cultivars, their location and growth conditions (soil, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The preferred transgenic plants or plant cultivars (obtained by genetic engineering) which are to be treated according to the invention include all plants which, by virtue of the genetic modification, received genetic material which imparts particularly advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products. Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape, and also fruit plants (with the fruits apples, pears, citrus fruits and grapes), and particular emphasis is given to maize, soya beans, potatoes, cotton, and oilseed rape. “Traits” that are emphasized are in particular increased defence of the plants against insects by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof) (hereinbelow referred to as “Bt plants”). Traits that are also particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potatoes). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plant cultivars will be developed and/or marketed in the future.

Moreover, the active compound combinations according to the invention can be used in the protection of materials for protecting industrial materials against unwanted fungi. Industrial materials are, for example, paper, carpets, buildings, cooling and heating systems, wall coverings, insulation and air conditioning units. The active compound combinations according to the invention may prevent disadvantageous effects, such as rotting, decay, discolouration, decolouration or formation of mould.

The method according to the invention for controlling unwanted fungi can also be employed for protecting storage goods. Here, storage goods are to be understood as meaning natural substances of vegetable or animal origin or processed products thereof of natural origin, for which long-term protection is desired. Storage goods of vegetable origin, such as, for example, plants or plant parts, such as stems, leaves, tubers, seeds, fruits, grains, can be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The active compound combinations according to the invention can prevent disadvantageous effects, such as rotting, decay, discolouration, decolouration or the formation of mould.

Some pathogens of fungal diseases which can be treated according to the invention may be mentioned by way of example, but not by way of limitation:

Diseases caused by powdery mildew pathogens, such as, for example, Blumeria species, such as, for example, Blumeria graminis; Podosphaera species, such as, for example, Podosphaera leucotricha; Sphaerotheca species, such as, for example, Sphaerotheca fuliginea; Uncinula species, such as, for example, Uncinula necator;
Diseases caused by rust disease pathogens, such as, for example, Gymnosporangium species, such as, for example, Gymnosporangium sabinae; Hemileia species, such as, for example, Hemileia vastatrix; Phakopsora species, such as, for example, Phakopsora pachyrhizi and Phakopsora meibomiae; Puccinia species, such as, for example, Puccinia recondita or Puccinia triticina; Uromyces species, such as, for example, Uromyces appendiculatus;
Diseases caused by pathogens from the group of the Oomycetes, such as, for example, Bremia species, such as, for example, Bremia lactucae; Peronospora species, such as, for example, Peronospora pisi or P. brassicae; Phytophthora species, such as, for example Phytophthora infestans; Plasmopara species, such as, for example, Plasmopara viticola; Pseudoperonospora species, such as, for example, Pseudoperonospora humuli or Pseudoperonospora cubensis; Pythium species, such as, for example, Pythium ultimum;
Leaf blotch diseases and leaf wilt diseases caused, for example, by Alternaria species, such as, for example, Alternaria solani; Cercospora species, such as, for example, Cercospora beticola; Cladiosporium species, such as, for example, Cladiosporium cucumerinum; Cochliobolus species, such as, for example, Cochliobolus sativus (conidia form: Drechslera, Syn: Helminthosporium); Colletotrichum species, such as, for example, Colletotrichum lindemuthanium; Cycloconium species, such as, for example, Cycloconium oleaginum; Diaporthe species, such as, for example, Diaporthe citri; Elsinoe species, such as, for example, Elsinoe fawcettii; Gloeosporium species, such as, for example, Gloeosporium laeticolor; Glomerella species, such as, for example, Glomerella cingulata; Guignardia species, such as, for example, Guignardia bidwelli; Leptosphaeria species, such as, for example, Leptosphaeria maculans; Magnaporthe species, such as, for example, Magnaporthe grisea; Microdochium species, such as, for example, Microdochium nivale; Mycosphaerella species, such as, for example, Mycosphaerella graminicola and M. fijiensis; Phaeosphaeria species, such as, for example, Phaeosphaeria nodorum; Pyrenophora species, such as, for example, Pyrenophora teres; Ramularia species, such as, for example, Ramularia collo-cygni; Rhynchosporium species, such as, for example, Rhynchosporium secalis; Septoria species, such as, for example, Septoria apii; Typhula species, such as, for example, Typhula incarnata; Venturia species, such as, for example, Venturia inaequalis;
Root and stem diseases caused, for example, by Corticium species, such as, for example, Corticium graminearum; Fusarium species, such as, for example, Fusarium oxysporum; Gaeumannomyces species, such as, for example, Gaeumannomyces graminis; Rhizoctonia species, such as, for example Rhizoctonia solani; Tapesia species, such as, for example, Tapesia acuformis; Thielaviopsis species, such as, for example, Thielaviopsis basicola;
Ear and panicle diseases (including maize cobs) caused, for example, by Alternaria species, such as, for example, Alternaria spp.; Aspergillus species, such as, for example, Aspergillus flavus; Cladosporium species, such as, for example, Cladosporium cladosporioides; Claviceps species, such as, for example, Claviceps purpurea; Fusarium species, such as, for example, Fusarium culmorum; Gibberella species, such as, for example, Gibberella zeae; Monographella species, such as, for example, Monographella nivalis; Septoria species, such as for example, Septoria nodorum;
Diseases caused by smut fungi, such as, for example, Sphacelotheca species, such as, for example, Sphacelotheca reiliana; Tilletia species, such as, for example, Tilletia caries; T. controversa; Urocystis species, such as, for example, Urocystis occulta; Ustilago species, such as, for example, Ustilago nuda; U. nuda tritici;
Fruit rot caused, for example, by Aspergillus species, such as, for example, Aspergillus flavus; Botrytis species, such as, for example, Botrytis cinerea; Penicillium species, such as, for example, Penicillium expansum and P. purpurogenum; Sclerotinia species, such as, for example, Sclerotinia sclerotiorum;
Verticilium species, such as, for example, Verticilium alboatrum;
Seed- and soil-borne rot and wilt diseases, and also diseases of seedlings, caused, for example, by Fusarium species, such as, for example, Fusarium culmorum; Phytophthora species, such as, for example, Phytophthora cactorum; Pythium species, such as, for example, Pythium ultimum; Rhizoctonia species, such as, for example, Rhizoctonia solani; Sclerotium species, such as, for example, Sclerotium rolfsii;
Cancerous diseases, galls and witches' broom caused, for example, by Nectria species, such as, for example, Nectria galligena;
Wilt diseases caused, for example, by Monilinia species, such as, for example, Monilinia laxa;
Deformations of leaves, flowers and fruits caused, for example, by Taphrina species, such as, for example, Taphrina deformans;
Degenerative diseases of woody plants caused, for example, by Esca species, such as, for example, Phaemoniella clamydospora and Phaeoacremonium aleophilum and Fomitiporia mediterranea;
Diseases of flowers and seeds caused, for example, by Botrytis species, such as, for example, Botrytis cinerea;
Diseases of plant tubers caused, for example, by Rhizoctonia species, such as, for example, Rhizoctonia solani; Helminthosporium species, such as, for example, Helminthosporium solani;
Diseases caused by bacteriopathogens, such as, for example, Xanthomonas species, such as, for example, Xanthomonas campestris pv. oryzae; Pseudomonas species, such as, for example, Pseudomonas syringae pv. lachrymans; Erwinia species, such as, for example, Erwinia amylovora.

Preference is given to controlling the following diseases of soya beans:

fungal diseases on leaves, stems, pods and seeds caused, for example, by alternaria leaf spot (Alternaria spec. atrans tenuissima), anthracnose (Colletotrichum gloeosporoides dematium var. truncatum), brown spot (Septoria glycines), cercospora leaf spot and blight (Cercospora choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi Phakopsora meibomiae), scab (Sphaceloma glycines), stemphylium leaf blight (Stemphylium botryosum), target spot (Corynespora cassiicola)
Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmopspora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia Southern blight (Sclerotinia rolfsii), thielaviopsis root rot (Thielaviopsis basicola).

The application rate of the active compound combinations according to the invention is
when treating leaves: from 0.1 to 10 000 g/ha, preferably from 10 to 1 000 g/ha, particularly preferably from 50 to 300 g/ha (when the application is carried out by watering or dripping, it may even be possible to reduce the application rate, in particular when inert substrates such as rock wool or perlite are used);
when treating the soil: from 0.1 to 10 000 g/ha, preferably from 1 to 5 000 g/ha.

These application rates are mentioned only by way of example and not by way of limitation in the sense of the invention.

The active compound combinations or compositions according to the invention can thus be employed for protecting plants for a certain period of time after treatment against attack by the pathogens mentioned. The period for which protection is provided extends generally for 1 to 28 days, preferably 1 to 14 days, after the treatment of the plants with the active compounds.

In addition, by the treatment according to the invention it is possible to reduce the mycotoxin content in the harvested material and the foodstuff and feedstuff prepared therefrom. Particular, but not exclusive, mention may be made here of the following mycotoxins: deoxynivalenol (DON), nivalenol, 15-Ac-DON, 3-Ac-DON, T2- and HT2-toxin, fumonisine, zearalenon, moniliformin, fusarin, diaceotoxyscirpenol (DAS), beauvericin, enniatin, fusaroproliferin, fusarenol, ochratoxins, patulin, ergot alkaloids and aflatoxins produced, for example, by the following fungi: Fusarium spec., such as Fusarium acuminatum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides, inter alia, and also by Aspergillus spec., Penicillium spec., Claviceps purpurea, Stachybotrys spec. inter alia.

The invention is illustrated by the examples below. However, the invention is not limited to the examples.

### EXAMPLE A

### Effect of Additives on the Plant Compatibility of Triazoles in the Case of Soya Beans

Soya bean plants were cultivated under greenhouse conditions. Two- to three-week-old plants were treated with spray liquors of the preparations mentioned below. To this end, the test formulations were in each case prepared in tap water, and the additive to be tested was added. After at least 10 min of stirring, the spray liquor was pipetted by hand as 10-μ1-drops to the adaxial side of adult leaves. Each leave was treated with a total of four drops, and, depending on the variant, 2-3 leaves were used (depending on the availability of plant material).

The evaluation of phytotoxicity was carried out over a period of 5 days and recorded in 4 classes by scoring. Here, a distinction is made between a phytotoxicity (PTX) of 0 without symptoms, a PTX 1 with slight discolorations, a PTX 2 with marked spot-like and/or circular, but not total necrosis, and PTX 3 with total necrosis.

The active compounds are used in the form of commercial formulations, tebuconazole as Folicur® EW 250 (contains 250 g/l of active compound), prothioconazole as Proline® EC 250 (contains 250 g/l of active compound) and the combination of tebuconazole and trifloxystrobin as Nativo SC 300 (contains 200 g/l of tebuconazole and 100 g/l of trifloxystrobin).

The sulphosuccinates are used either in a solid application form as Aerosol OT-B or in liquid application form as Triton GR-7ME.

Other well tolerated commercial additives are used for comparison:

Agrho DR2000 (a polymeric hydroxypropyl derivative of Guar), emulsifier PS16 [a nonionic surfactant, tris(1-phenylethyl)phenol ethoxylate], Dash HC (a mixture of aromatic hydrocarbons, unsaturated fatty acids, methyl oleate and emulsifiers), Surfon 8515 [methyl oleate (about 90%) and emulsifiers).

| Formulation (in each | | | |
|---|---|---|---|
| case at 0.5 g/l of | | Additive | |
| tebuconazole or | | concentration | Phytotoxicity |
| prothioconazole) | Additive | (g/l) | (0-3)* |
| Folicur EW250 | — | — | 3 |
| Folicur EW250 | Aerosol OT-B | 0.1 | 1 |
| Folicur EW250 | Aerosol OT-B | 0.3 | 0 |
| Folicur EW250 | Aerosol OT-B | 1.0 | 0 |
| Folicur EW250 | Triton GR-7ME | 0.1 | 1 |
| Folicur EW250 | Triton GR-7ME | 0.3 | 1 |
| Folicur EW250 | Triton GR-7ME | 1.0 | 0 |
| Folicur EW250 | Agrho DR2000 | 1 | 3 |
| Folicur EW250 | Emulsifier PS16 | 1 | 3 |
| Proline EC250 | — | — | 3 |
| Proline EC250 | Triton GR-7ME | 0.1 | 1 |
| Proline EC250 | Triton GR-7ME | 0.3 | 1 |
| Proline EC250 | Triton GR-7ME | 1.0 | 0 |
| Proline EC250 | Triton GR-7ME | 3.0 | 0 |
| Proline EC250 | Aerosol OT-B | 1.0 | 0 |
| Proline EC250 | Dash HC | 3.0 | 3 |
| Nativo SC300 | — | — | 0 |
| Nativo SC300 | Aerosol OT-B | 1 g/l | 0 |
| Nativo SC300 | Dash HC | 3 g/l | 3 |
| Nativo SC300 | Surfon 8515 | 1 g/l | 3 |

*Ranking 0-3 over a period of up to 5 days after foliar application of individually 10-μl-drops
0: No change, as control,
1: Slight change
2: Marked necrotic spots or circles, but not over the entire area
3: Total necrosis

1) A method for reducing the phytotoxicity of triazole fungicides on dicotyledonous plants, comprising adding to a fungicidal composition comprising at least one triazole fungicide, one or more compounds of formula (I) 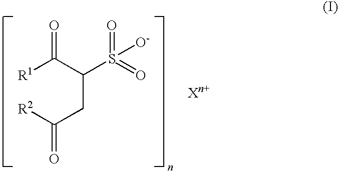
in which
R¹ and R² independently of one another represent hydroxy, alkoxy or cycloalkoxy or represent the group 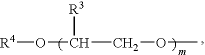
where at least one radical R¹ or R² does not represent hydroxy,
R³ represents hydrogen or methyl,
R⁴ represents hydrogen, alkyl or aryl,
m represents an integer from 0 to 100,
X represents an alkali metal, where n represents 1, or an alkaline earth metal, where n represents 2.

2) The method according to claim 1, wherein the compound of the formula (I) is selected from the group consisting of
sodium diisopropylsulphosuccinate,
sodium diisobutylsulphosuccinate,
sodium dipentylsulphosuccinate,
sodium dihexylsulphosuccinate,
sodium dioctylsulphosuccinate,
sodium di(2-ethylhexyl)sulphosuccinate,
sodium didodecylsulphosuccinate,
sodium tridecylsulphosuccinate, and
sodium dicyclohexylsulphosuccinate.

3) The method according to claim 1, wherein the fungicidal composition is a ready-to-use spray liquor, and wherein the one or more compounds of formula (I) are present in a concentration of from 0.05 to 5 g/l.

4) The method according to claim 1, wherein the at least one triazole fungicide is selected from the group consisting of azaconazole, bitertanol, bromuconazole, cyproconazole, difenoconazole, diniconazole, diniconazole-M, epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, simeconazole, tebuconazole, tetraconazole, triadimefon, triadimenol, and triticonazole, or a salt thereof.

5) The method according to claim 1, wherein the fungicidal composition comprises a binary or ternary mixture of triazole fungicides or a mixture of one or more triazole fungicides with one or more inhibitors at complex III of the respiratory chain.

6) The method according to claim 1, wherein the at least one triazole fungicide is a single active compound selected from the group consisting of cyproconazole, epoxiconazole, metconazole, prothioconazole and tebuconazole; or a mixture selected from the group consisting of tebuconazole and prothioconazole, tebuconazole and epoxiconazole, prothioconazole and epoxiconazole, tebuconazole and trifloxystrobin, tebuconazole and fluoxastrobin, tebuconazole and azoxystrobin, tebuconazole and pyraclostrobin, prothioconazole and trifloxystrobin, prothioconazole and azoxystrobin, and also prothioconazole and pyraclostrobin.

7-12. (canceled)

13) A composition, comprising
(a) a triazole fungicide and
(b) one or more compounds of formula (I) 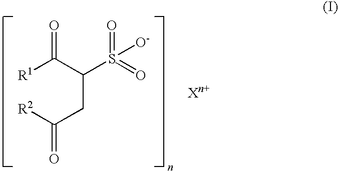
in which
R¹ and R² independently of one another represent hydroxy, alkoxy or cycloalkoxy or represent the group 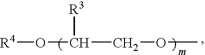
where at least one radical R¹ or R² does not represent hydroxy,
R³ represents hydrogen or methyl,
R⁴ represents hydrogen, alkyl or aryl,
m represents an integer from 0 to 100,
X represents an alkali metal, where n represents 1, or an alkaline earth metal, where n represents 2.

14) The composition according to claim 13, wherein the compound of formula (I) is selected from the group consisting of
sodium diisopropylsulphosuccinate,
sodium diisobutylsulphosuccinate,
sodium dipentylsulphosuccinate,
sodium dihexylsulphosuccinate,
sodium dioctylsulphosuccinate,
sodium di(2-ethylhexyl)sulphosuccinate,
sodium didodecylsulphosuccinate,
sodium tridecylsulphosuccinate, and
sodium dicyclohexylsulphosuccinate.

15) The composition according to claim 13, comprising a triazole fungicide selected from the group consisting of azaconazole, bromuconazole, cyproconazole, difenoconazole, diniconazole, diniconazole-M, epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, simeconazole, tetraconazole, triadimefon, triadimenol, and triticonazole, or a salt thereof.

16) The composition according to claim 13, comprising from 0.05 to 10 parts by weight of the one or more compounds of formula (I) per part by weight of the triazole fungicide.

17) The composition according to claim 13, comprising a binary or ternary mixture of triazole fungicides or a mixture of one or more triazole fungicides with one or more inhibitors at complex III of the respiratory chain.

18) The composition according to claim 13, comprising an individual active compound selected from the group consisting of cyproconazole, epoxiconazole, metconazole, prothioconazole; or a mixture selected from the group consisting of tebuconazole and prothioconazole, tebuconazole and epoxiconazole, prothioconazole and epoxiconazole, tebuconazole and trifloxystrobin, tebuconazole and fluoxastrobin, tebuconazole and azoxystrobin, tebuconazole and pyraclostrobin, prothioconazole and trifloxystrobin, prothioconazole and azoxystrobin and also prothioconazole and pyraclostrobin.

19) The method according to claim 2, wherein the compound of formula (I) is sodium di(2-ethylhexyl)sulphosuccinate.

20) The method according to claim 5, wherein the inhibitor is a strobilurin.

21) The composition according to claim 14, wherein the compound of formula (I) is sodium di(2-ethylhexyl)sulphosuccinate

22) The composition according to claim 16, comprising from 0.1 to 2 parts by weight of the one or more compounds of formula (I) per part by weight of the triazole fungicide.

23) The composition according to claim 22, comprising from 0.2 to 1 parts by weight of the one or more compounds of formula (I) per part by weight of the triazole fungicide.

24) The composition according to claim 17, wherein the inhibitor is a strobilurin.
